(19) United States
(12) Patent Application Publication
(10) Pub. No.: US 2020/0411171 A1
(43) Pub. Date: Dec. 31, 2020

(54) COMPUTER SYSTEM AND METHOD FOR WORKLIST PRIORITIZATION FOR CLINICAL DOCUMENTATION IMPROVEMENT (CDI) IN MEDICAL CODING

(71) Applicant: ezDI INC, Louisville, KY (US)

(72) Inventors: Nehal Shah, Louisville, KY (US); Raxitkumar Vishnupuri Goswami, Gujarat (IN); Suhas Indirakshan Nair, Gujarat (IN); Vatsal Nareshkumar Shah, Gujarat (IN); Vivek Kumar, Gujarat (IN)

(21) Appl. No.: 16/911,861

(22) Filed: Jun. 25, 2020

Related U.S. Application Data
(60) Provisional application No. 62/866,154, filed on Jun. 25, 2019.

(51) Int. Cl. G16H 40/20; G16H 70/20; G16H 50/20; G16H 50/70; G06Q 10/06; G16H 10/60; H04L 29/06; G06F 16/9535; G06F 16/25; G06F 16/242; G06F 16/2457; G06F 40/205

(52) U.S. Cl. CPC G16H 40/20; G16H 70/20; G16H 50/20; G16H 50/70; G06Q 10/06316; G06Q 10/063112; G06F 40/205; H04L 63/04; G06F 16/9535; G06F 16/258; G06F 16/243; G06F 16/24578; G16H 10/60

(57) ABSTRACT

A method for worklist prioritization for Clinical Documentation Improvement (CDI) in medical coding comprises receiving one or more cases from an admin computing device associated with a hospital administration, wherein each of the one or more cases is assigned a predetermined weightage to a corresponding plurality of parameters involved in each case; generating a confidence score of each of the one or more cases; adding the predetermined weightages of each of the one or more cases based on the confidence score; providing the one or more cases in a sequence based on a sum of predetermined weightages of each of the one or more cases from highest to lowest; and marking & scheduling the one or more cases in the generated sequence for a CDI Specialist (CDS) for review and take up of the one or more case based on the priority level for query generation.

### TECHNICAL FIELD

The present invention relates to implementations of Clinical Documentation Improvement (CDI) and more particularly to a computer system and method for worklist prioritization for CDI in medical coding.

### BACKGROUND OF THE INVENTION

Recent changes in Medicare coding requirements have caused hospitals to suffer from lost revenues, penalties and forfeiture of reimbursements due to inadequate documentation. The role of CDI programs continues to evolve, driven mainly by a focus on improving quality care, reimbursement, and reporting. CDI is the consistent improvement not only in the document but also in the information processing and management processes in a clinical situation. CDI programs require Physicians, Nurses, Pharmacists, and health information specialists to work together because CDI includes various care processes such as medical procedures, nursing care, laboratory work, rehabilitation, etc.

The success of CDI programs lies in integrating people, processes and technology in order to provide the specificity of documentation required by ICD-10, the meaningful use as well as other quality care initiatives. The importance of accurate clinical documentation cannot be understated and is no longer a low-level priority for healthcare facilities today. It is a vital component to patient care, physician satisfaction, and revenue cycle strategies. CDI specialists, along with clinical care providers and administration must contribute to organizational success and ensure the right information is available at the right time.

Since 1928, AHIMA has recognized that clinical data and information is a critical resource needed for efficacious healthcare. Health Information Management professionals strive to ensure that healthcare information used during patient care is valid, accurate, complete, trustworthy, and timely. But current healthcare industry pressures are demanding change. Hospitals and providers must improve clinical documentation in preparation for the expanded scope of clinical data beyond a single patient encounter to a comprehensive data set comprising the entire continuum of care, a concept that will become monumental with the specificity required with the impending implementation of ICD-10 coding classification system in October 2015.

As healthcare reform moves quickly towards quality-driven reimbursement, organizations and providers continue to justify care plans and treatment options as well as successfully demonstrate quality outcomes and patient safety. Consistent, complete, and accurate documentation is the key to the economic health of the organization and a key indicator of physician quality. Organizations and providers need to be able to use automated, intuitive tools to successfully implement new technology, new federal requirements, and specific strategic initiatives without compromising patient care. All quality metrics for any hospital are interlinked to each other. However, the single and most important thing that connects all of them is documentation. Everything starts with and is affected by the quality of documentation present in the patient's record. The more complete and accurate documentation, the better all metrics will be, and the better the hospital's revenue. A good program becomes the mainstay of the hospital, helping to link and connect all aspects of care delivered to every patient during admission.

Presently, the CDI worklists are based on payor, floor unit etc. The CDI specialist (CDS) reviews 100 percent of the parameters for the target floor, payor etc. All the reviews by the CDS do not result in query or documentation improvement. This is very time consuming and at times the quality of the review may also be compromised due to workload. One more drawback of the currently used CDI worklists is that it lacks specificity.

Therefore there is a need in the art for a computer system and method for worklist prioritization for clinical documentation improvement (CDI) in medical coding that takes care of these issues by prioritizing the CDI worklist and allowing the CDS to focus on the more important and urgent cases.

### SUMMARY OF THE INVENTION

Embodiments of the present invention aim to provide a computer system and method for worklist prioritization for CDI in medical coding that involves assigning desired weightages to cases based on different parameters like Severity of illness, length of stay, clinical validation, 30 days readmission etc. Once the weightages are assigned, the cases are sequenced as per the weightages from higher to lower weightage, so that the CDI specialist can prioritize and concentrate on the most important cases. Moreover, these parameters can be modified or customized as per the hospital requirements.

According to a first aspect of the present invention, there is provided a computer system for worklist prioritization for Clinical Documentation Improvement (CDI) in medical coding. The computer system comprises, but not limited to, a memory unit configured to store machine-readable instructions; and a processor operably connected with the memory unit. Further, the processor obtains the machine-readable instructions from the memory unit, and is configured by the machine-readable instructions to receive one or more cases from an admin computing device associated with a hospital administration, wherein each of the one or more cases is assigned a predetermined weightage to a corresponding plurality of parameters involved in each case; generate a confidence score of each of the one or more cases to validate the one or more cases and the predetermined weightage assigned to a corresponding plurality of parameters involved in each case; add the predetermined weightages of each of the one or more cases based on the confidence score; provide the one or more cases in a sequence based on a sum of predetermined weightages of each of the one or more cases from highest to lowest, the highest being indicative of a high priority case; and mark & schedule the one or more cases in the generated sequence for a CDI Specialist (CDS) for review and take up based on the priority level for query generation.

In accordance with an embodiment of the present invention, for generating the confidence score, the processor is further configured to establish a secure interface two-way channel for data transfer between the computer system and the admin computing system; receive data related to the one or more cases from the admin computing device using the ecure interface two-way channel; segregate the data into text data and demographic data using a HL7 parser, the text data being unstructured patient-oriented clinical data; send the demographic data to an application database that stores all data of the one or more cases in one place from where a connected web service fetches information to send and receive client specific data; convert the text data using Natural Language Programming (NLP) from the unstructured data into structured data; build a query module using a query parser by receiving the text data from the NLP and a query authoring tool operated by a user, the query module being used to validate the one or more cases; pass the data from the query parser through a scheduler which is defined by the user and/or set of algorithms whenever a predetermined set of conditions is met to prioritize the CDI worklist; and receive the parsed query from the query parser and the data from the web service at a CDI worklist prioritization module, to generate the confidence score based on a defined algorithm.

In accordance with an embodiment of the present invention, the review is selected from an initial review and a follow up review.

In accordance with an embodiment of the present invention, the plurality of parameters for the initial review are selected from one or more of DRG Impacting Query Opportunity, Risk of mortality, Quality Impacting Query Opportunity, Target Chief Complaint/Admitting Diagnosis, Clinical Validation (Missing Diagnosis and missing evidence), PSI Flag, All Mortalities, No Major Comorbidity/Complication (MCC), 30-day readmission, Denials, Target Diagnosis Related Group (DRG), Target Principal/Primary Diagnosis, Assigned by Coding, Assigned by Quality and standard review.

In accordance with an embodiment of the present invention, the plurality of parameters for the follow-up review are selected from one or more of Patient Expired, Discharged with pending queries, Query Responded, New DRG Impacting Query Opportunity, New Quality Impacting Query Opportunity, Scheduled for Today, DRG Mismatch, geometric mean length of stay (GMLOS), Missing documents received, New documents received, On Hold—Pending Queries, On Hold—No Queries and Awaiting Reconciliation.

In accordance with an embodiment of the present invention, the predetermined weightages are provided on a scale of 1 to 10, wherein 10 is highest & indicative of higher priority.

According to a second aspect of the present invention, there is provided a method for worklist prioritization for Clinical Documentation Improvement (CDI) in medical coding. The method comprises receiving one or more cases from an admin computing device associated with a hospital administration, wherein each of the one or more cases is assigned a predetermined weightage to a corresponding plurality of parameters involved in each case; generating a confidence score of each of the one or more cases to validate the one or more cases and the predetermined weightage assigned to a corresponding plurality of parameters involved in each case; adding the predetermined weightages of each of the one or more cases based on the confidence score; providing the one or more cases in a sequence based on a sum of predetermined weightages of each of the one or more cases from highest to lowest, the highest being indicative of a high priority case; and marking & scheduling the one or more case in the generated sequence for a CDI Specialist (CDS) for review and take up of the one or more case based on the priority level for query generation.

In accordance with an embodiment of the present invention, for generating the confidence score, the method further comprises the steps of establishing a secure interface two-way channel for data transfer between the computer system and the admin computing system; receiving data related to the one or more cases from the admin computing device using the secure interface two-way channel; segregating the data into text data and demographic data using a HL7 parser, the text data being unstructured patient-oriented clinical data; sending the demographic data to an application database that stores all data of the one or more cases in one place from where a connected web service fetches information to send and receive client specific data; converting the text data using Natural Language Processing (NLP) from the unstructured data into structured data; building a query module using a query parser by receiving the text data from the NLP and a query authoring tool operated by a user, the query module being used to validate the one or more cases; passing the data from the query parser through a scheduler which is defined by the user and/or set of algorithms whenever a predetermined set of conditions is met to prioritize the CDI worklist; and receiving the parsed query from the query parser and the data from the web service at a CDI worklist prioritization module, to generate the confidence score based on a defined algorithm.

In accordance with an embodiment of the present invention, the review is selected from an initial review and a follow up review.

In accordance with an embodiment of the present invention, the plurality of parameters for the initial review are selected from one or more of DRG Impacting Query Opportunity, Risk of mortality, Quality Impacting Query Opportunity, Target Chief Complaint/Admitting Diagnosis, Clinical Validation (Missing Diagnosis and missing evidence), PSI Flag, All Mortalities, No Major Comorbidity/Complication (MCC), 30-day readmission, Denials, Target Diagnosis Related Group (DRG), Target Principal/Primary Diagnosis, Assigned by Coding, Assigned by Quality and standard review.

In accordance with an embodiment of the present invention, the plurality of parameters for the follow-up review are selected from one or more of Patient Expired, Discharged with pending queries, Query Responded, New DRG Impacting Query Opportunity, New Quality Impacting Query Opportunity, Scheduled for Today, DRG Mismatch, geometric mean length of stay (GMLOS), Missing documents received, New documents received, On Hold—Pending Queries, On Hold—No Queries and Awaiting Reconciliation.

In accordance with an embodiment of the present invention, the predetermined weightages are provided on a scale of 1 to 10, wherein 10 is highest & indicative of higher priority.

### BRIEF DESCRIPTION OF DRAWINGS

So that the manner in which the above recited features of the present invention can be understood in detail, a more particular description of the invention, briefly summarized above, may have been referred by embodiments, some of which are illustrated in the appended drawings. It is to be noted, however, that the appended drawings illustrate only typical embodiments of this invention and are therefore not to be considered limiting of its scope, for the invention may admit to other equally effective embodiments.

These and other features, benefits, and advantages of the present invention will become apparent by reference to the following text figure, with like reference numbers referring to like structures across the views, wherein:

FIG. 1 is an exemplary environment of computing devices to which the various embodiments described herein may be implemented;

FIG. 2 illustrates a method for worklist prioritization for clinical documentation improvement (CDI) in medical coding, in accordance with an embodiment of the present invention; and

FIG. 3 illustrates an information flow diagram for measuring/generating a confidence score, in accordance with an embodiment of the present invention.

### DETAILED DESCRIPTION OF EMBODIMENTS

The present invention is described hereinafter by various embodiments with reference to the accompanying drawing, wherein reference numerals used in the accompanying drawing correspond to the like elements throughout the description.

While the present invention is described herein by way of example using embodiments and illustrative drawings, those skilled in the art will recognize that the invention is not limited to the embodiments of drawing or drawings described and are not intended to represent the scale of the various components. Further, some components that may form a part of the invention may not be illustrated in certain figures, for ease of illustration, and such omissions do not limit the embodiments outlined in any way. It should be understood that the drawings and detailed description thereto are not intended to limit the invention to the particular form disclosed, but on the contrary, the invention is to cover all modifications, equivalents, and alternatives falling within the scope of the present invention as defined by the appended claim. As used throughout this description, the word “may” is used in a permissive sense (i.e. meaning having the potential to), rather than the mandatory sense, (i.e. meaning must). Further, the words “a” or “an” mean “at least one” and the word “plurality” means “one or more” unless otherwise mentioned. Furthermore, the terminology and phraseology used herein is solely used for descriptive purposes and should not be construed as limiting in scope. Language such as “including,” “comprising,” “having,” “containing,” or “involving,” and variations thereof, is intended to be broad and encompass the subject matter listed thereafter, equivalents, and additional subject matter not recited, and is not intended to exclude other additives, components, integers or steps. Likewise, the term “comprising” is considered synonymous with the terms “including” or “containing” for applicable legal purposes.

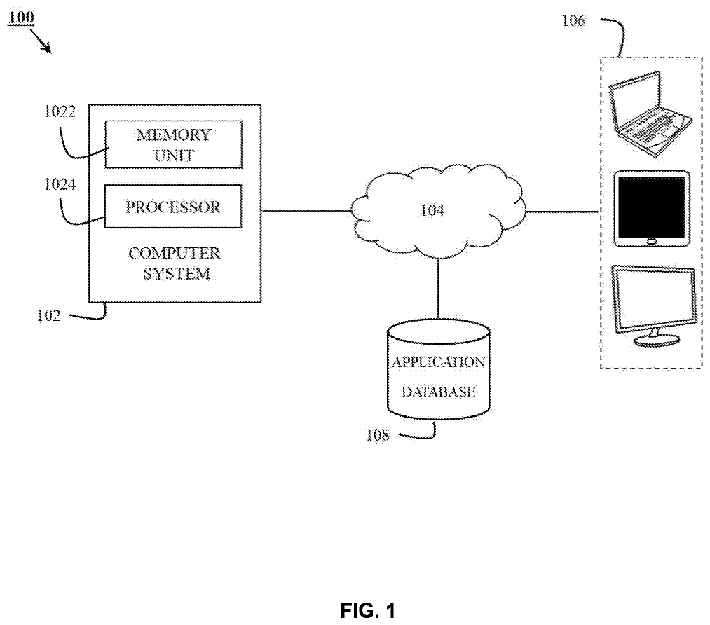

Referring to the drawings, the invention will now be described in more detail. FIG. 1 illustrates an exemplary environment 100 of computing devices to which the various embodiments described herein may be implemented. The environment comprises a computer system 102 connected with an admin computing device 106 via a network 104.

Herein the admin computing device 106 is envisaged to be associated with a hospital administration that receives the one or more cases (medical cases). The hospital administration is envisaged to assign a predetermined weightage to a plurality of parameters associated with each case using the admin computing device 106. Some exemplary plurality of parameters may be, but not limited to, Severity of illness, length of stay, clinical validation, 30 days readmission etc. Theses parameters and along with other specific parameters will be discussed in detail later in the description. Accordingly, the admin computing device 106 is selected from a group comprising, but not limited to, a laptop, a desktop, PDA and a portable handheld device such as a smartphone or a tablet, having computing capabilities and comprising at least a display module, an input module and a user interface.

Further, the network 104 may be one of, but not limited to, a Local Area Network (LAN) or a Wide Area Network (WAN) and may be implemented using a number of protocols, such as but not limited to, TCP/IP, 3GPP, 3GPP2, LTE, IEEE 802.x, HTTP, HTTPS, UDP, RTMP etc. One would appreciate that the network 104 can be a short-range communication network and/or a long-range communication network. In one embodiment, the network 104 may be wireless intranet network that does not require web connectivity. In another embodiment, the network 104 is internet.

Further as shown in FIG. 1, the environment further comprises the computer system 102. The computer system 102 may connected with the admin computing device 106 via the network 104, by any suitable means, such as, for example, hardwired and/or wireless connections, such as dial-up, hardwired, cable, Digital Subscriber Line (DSL), satellite, cellular, Personal Communications Service (PCS), wireless transmission. The computer system 102 may be associated with a CDI specialist (CDS). The computer system 102 may be a portable computing device, a desktop computer or a server stack.

The computer system 102 is envisaged to include computing capabilities such as a memory unit 1022 configured to store machine readable instructions. The machine-readable instructions may be loaded into the memory unit 1022 from a non-transitory machine-readable medium such as, but not limited to, CD-ROMs, DVD-ROMs and Flash Drives. Alternately, the machine-readable instructions may be loaded in a form of a computer software program into the memory unit 1022. The memory unit 1022 in that manner may be selected from a group comprising EPROM, EEPROM and Flash memory. Further, the computer system 102 includes a processor 1024 operably connected with the memory unit 1022. In various embodiments, the processor 1024 is one of, but not limited to, a general-purpose processor, an application specific integrated circuit (ASIC) and a field-programmable gate array (FPGA).

The computer system 102 is envisaged to be connected with a display means and an input means. The display means is an output device configured to represent data/content in human understandable form. Therefore, the display means is one of, but not limited to, CRT, TFT, LCD, LED, OLED and AMOLED display. Also, the input means is one of, but not limited to, a keyboard, a mouse, a touchpad or a trackball. In embodiment, the processor 1024 is configured to implement Artificial Intelligence (AI), Machine Learning (ML) and deep learning technologies for, but not limited to, data analysis, collating data & presentation of data in real-time.

In one embodiment, the computer system 102 further includes different modules and techniques to generate confidence score based on the plurality of parameters. In one embodiment, the computer system 102 may include (not shown in FIG. 1), but not limited to, a HL7 parser, a query parser, Natural Language Programming and a CDI prioritization module. Additionally, a machine learning pipeline may be built starting from the point of collecting data to the point of generating confidence score. The modules may include the following components, but not limited to, collector module, Pre-Processor/Cleaner, Model Builder, Score Generator etc.

In accordance with an embodiment of the present invention, an application database 108 is also connected with the network 104. The application database 108 may be a local or a cloud-based storage. The application database 108 is configured to store all data of the one or more cases in one place from where a connected web service may fetch information to send and receive client specific data.

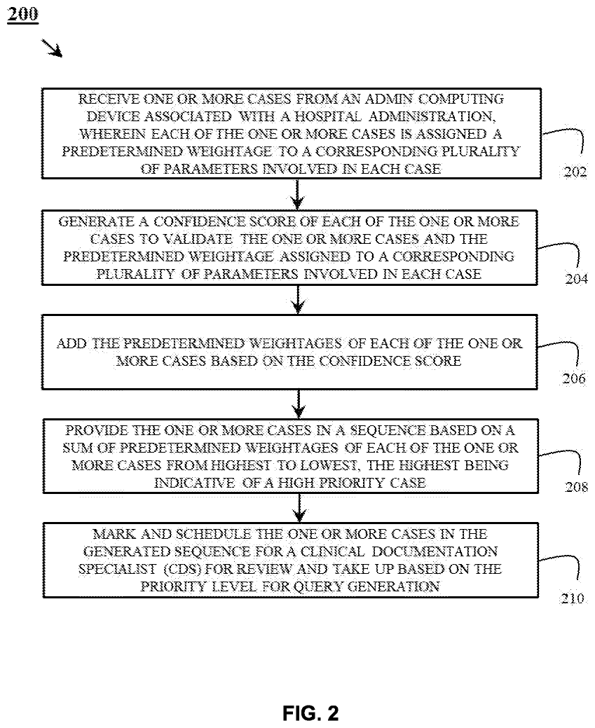

The present invention may be implemented using the following method 200, (without limiting to any particular order of steps). FIG. 2 illustrates a method 200 for worklist prioritization for Clinical Documentation Improvement (CDI) in medical coding, in accordance with an embodiment of the present invention. As shown in FIG. 2, the method 200 starts at step 202 by receiving one or more cases at the computer system 102 from the admin computing device 106 associated with the hospital administration. As previously mentioned, the hospital administration is envisaged to assign a predetermined weightage to the corresponding plurality of parameters involved in each case. The one or more parameters may be, but not limited to, Severity of illness, length of stay, clinical validation, 30 days readmission etc. The one or more parameters are customizable and the weightage assigned to each parameter can also be modified as per the hospital needs. For the present example, it is assumed that weightage ranges from 1-10, where 10 is the highest.

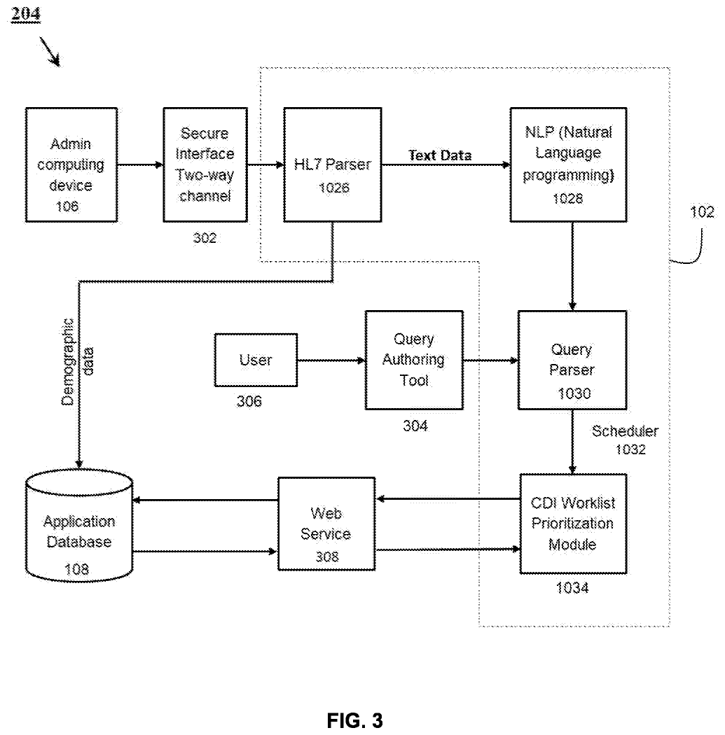

Then at step 204, the processor 1024 is configured to generate a confidence score of each of the one or more cases to validate the one or more cases and the predetermined weightage assigned to a corresponding plurality of parameters involved in each case. In simple terms, confidence scores indicate the authenticity of the case and weightages assigned. FIG. 3 illustrates an information flow diagram for measuring/generating a confidence score, in accordance with an embodiment of the present invention. As shown in FIG. 3, the computer system 102 is envisaged to further include the HL7 parser 1026, the Natural Language Programming (NLP 1028), the query parser 1030, a scheduler 1032 and the CDI worklist prioritization 1034.

The admin computing device 106 is envisaged to be associated with the hospital administration facility i.e. the client using the services to run the facility or hospital and entire cases or medical documents data is stored at this location. Subsequently, the data is sent through a secure interface two-way channel 302 to the computer system 102 and is received at the HL7 parser. The secure interface two-way channel 302 ensures security of the data during the data transfer between the computer system 102 and the admin computing device 106. Then, the data is passed through the HL7 parser 1026, where the data is segregated into “Text Data” and “Demographic Data”. The text data may be, but not limited to, unstructured patient-oriented clinical data and the demographic graphic data may be personal details of the patient such as, but not limited to, birth date, age, location, date of admission in hospital etc. Further, the demographic data is sent to the application database 108 which is a collection of all client's data in one place from where a web service 308 can fetch the information to send and receive client specific data. The text data from HL7 parser 1026 is sent to NLP 1028 to convert the unstructured data into structured data. Further the query parser 1030 receives the text data from NLP 1028 and a query authoring tool 304 by user to build a query module. This module is used for validating the received cases. Here, the query authoring tool 304 is used to reduce the operating gap between the user 306 and the interface. This is a user-friendly tool wherein the user 306 with no prior knowledge on the specific ontology or programming query language skills can be used to customize the query according to the information needed. This is the tool wherein the user 306 has the flexibility to modify the query to get the desired outcome.

After that, the data from the query parser 1030 is passed through the scheduler 1032 (201), which is defined by the user 306 or set of algorithm whenever a certain set of condition is met to prioritize the CDI worklist. For example, CDI prioritisation can be done daily, weekly, or monthly or as per the user requirement. In another example: the scheduler 1032 may keep track of the patient's admission day and accordingly update and send the plurality of parameters after every predetermined no. of days, such as every 2 days, 5 days etc. In that sense, it will be appreciated by a skilled addressee that the scheduler 1032 is not necessarily operating between the query parser 1030. After that, the parsed query from the query parser and the data from the web service at the CDI worklist prioritization module 1034 of the computer system 102 to generate the confidence score based on a defined algorithm. In one exemplary embodiment, the confidence score may be a sum of selected one or more parameters out of the plurality of parameters depending on the type of review being done i.e. initial review or follow up review. However, it will be appreciated by a skilled addressee that other techniques that require further computation may also be implemented for generation of confidence scores, without departing from the scope of the present invention.

Returning to FIG. 2, after the generation of confidence scores, at step 206 the processor 1024 is configured to add the predetermined weightages of each of the one or more cases based on the confidence score. Onwards, at step 208, the processor 1024 is configured to provide the one or more cases in a sequence based on the on a sum of predetermined weightages of each of the one or more cases from highest to lowest. The highest weightage of any particular case indicates that it is a high priority case. So, the processor 1024 arranges the one or more in the order of priority. Additionally, at step 210, the processor 1024 marks and schedules the one or more case in the generated sequence for a CDI Specialist (CDS) for review and take up of the one or more case based on the priority level for query generation and documentation improvement. Herein the processor 1024 makes it easier for a CDS to know which cases to review and take up based on the order of priority. It is something which was completely missing from the prior art. The present invention enables the CDS to prioritize and concentrate on the most important cases.

Herein, the review may be an initial review and a follow-up review. So, there may be different plurality of parameters for each of the initial review or follow-up review, which will now be explained below.

In accordance with an embodiment of the present invention, the hospital administration selects a case for the initial review to assign the appropriate weightage for the plurality of parameters. The plurality of parameters that can be chosen by the hospital admin are mentioned below:

DRG Impacting Query Opportunity
Updating the diagnosis code in the document that impacts the DRG.

Risk of Mortality
The risk of mortality (ROM) provides a medical classification to estimate the likelihood of in hospital death for a patient. The ROM classes are minor (1), moderate (2), major (3), and extreme (4). The ROM class is used for the evaluation of patient mortality.

Quality Impacting Query Opportunity
These are the queries that have an impact on the parameters—Severity of Illness and Rate of Mortality.

Target Chief Complaint/Admitting Diagnosis
This is defined as the code associated with the diagnosis established at the time of the patient's admission to the hospital. It is the present on admission (POA) which is determined as the reason the admission.

Clinical Validation (Missing Diagnosis and Missing Evidence)
Clinical validation means that diagnoses documented in a patient's record is substantiated by clinical criteria generally accepted by the medical community. Generally accepted clinical criteria typically come from authoritative professional guidelines, consensus, or evidence-based sources.
   In the absence of such sources, a less objective test of clinical validity may be the clinical diagnostic standards that most clinicians in a comparable specialty would reasonably agree are sufficient for establishing a particular diagnosis. To better understand the concept of clinical validation, let's take a look at some specific examples.
   In its 2013 guideline, the American College of Gastroenterology said that the diagnosis of acute pancreatitis is most often established by the presence of 2 of the 3 criteria: abdominal pain consistent with the disease, serum amylase and/or lipase level greater than 3 times the upper limit of normal and characteristic findings from abdominal imaging. Clinical validation of acute pancreatitis would typically require at least 2 of these findings confirmed in the medical record unless the clinician documented a plausible alternative basis for the diagnosis that other clinicians would find reasonable.

PSI Flag
The Patient Safety Indicators (PSIs) are a set of indicators providing information on potential in hospital complications and adverse events following surgeries, procedures, and childbirth. The PSIs are used to help hospitals identify potential adverse events that might need further study, provide the opportunity to assess the incidence of adverse events and in hospital complications using administrative data found in the typical discharge record; include indicators for complications occurring in hospital that may represent patient safety events; and, indicators also have area level analogues designed to detect patient safety events on a regional level

All Mortalities
All mortalities are defined as the no. of death of patient(s) occurring in the hospital.

No CC/MCC
Major Comorbidity/Complication (MCC) is defined as the highest degree of severity of illness.
Comorbidity/Complication (CC)—this is the next degree of severity of illness; and No comorbidity/Complication—this does not in any significant degree affect the severity of illness or resource consumption

30-Day Readmission
All the unplanned readmissions that happen within 30 days of discharge from the initial admission. Patients who are readmitted to the same hospital, or another applicable acute care hospital for any reason.

Denials
Denials occur due to a lack of documentation or clinical evidence. The involvement of CDI professionals in the denials process can assist denials specialists in identifying appeals opportunities. CDI professionals can also incorporate the reasons for denials into their daily health record documentation reviews.

Target Diagnosis Related Group (DRG)
DRGs categorize patients with respect to diagnosis, treatment and length of hospital stay. The assignment of a DRG depends on the following variables: Principal diagnosis, Secondary diagnosis(es), Surgical procedures performed, Comorbidities and complications, Patient's age and sex, Discharge status

Target PDx
The Principal/Primary Diagnosis is the condition established after study to be mainly responsible for occasioning the admission of the patient to the hospital for care. Since the Principal/Primary Diagnosis represents the reason for the patient's stay, it may not necessarily be the diagnosis which represents the greatest length of stay, the greatest consumption of hospital resources, or the most life-threatening condition. Since the Principal/Primary Diagnosis reflects clinical findings discovered during the patient's stay, it may differ from Admitting Diagnosis.

Assigned by Coding
The assignment of a diagnosis code is based on the provider's diagnostic statement that the condition exists. The provider's statement that the patient has a particular condition is sufficient. Code assignment is not based on clinical criteria used by the provider to establish the diagnosis.

Assigned by Quality
The parameter wherein the case is tagged as marked as Assigned by quality i.e. there is no discrepancy between the assigned and accepted codes

Standard Review (No Flags)
The CDI review done as per the current accepted standards.

Whereas, the plurality of parameters for the follow up review are:

Patient Expired
When the patient dies in the hospital

Discharged with Pending Queries
When the patient is allowed to go home but there are some unanswered queries by the physician. It may be either.

Query Responded
This refers to the cases wherein the query raised by the coder is responded by the attending physician.

New DRG Impacting Query Opportunity
Updating the diagnosis code in the document that impacts the DRG.

New Quality Impacting Query Opportunity
The queries that impact SOI and ROM

Scheduled for Today
The cases that are scheduled to be reviewed the same day.

DRG Mismatch
A diagnosis-related group (DRG) is a patient classification system that standardizes prospective payment to hospitals and encourages cost containment initiatives. In general, a DRG payment covers all charges associated with an inpatient stay from the time of admission to discharge. The DRG includes any services performed by an outside provider. Claims for the inpatient stay are submitted and processed for payment only upon discharge. There are cases where there is an inconsistency between the DRG and the actual code.

LOS>Working GMLOS

Identifying when the Length of Stay is More than the GMLOS.
The goal of this quality improvement project is to reduce the length of hospitalization, to improve patient satisfaction and meet the geometric mean length of stay (GMLOS). At baseline, only 61 percent of patients met GMLOS. The project goal was to track and monitor current length of stay (LOS) and to increase the percentage of patients meeting GMLOS by 10 percent.
LOS greater than or equal to GMLOS (Medical Necessity Excluded)—The purpose of this MS-DRG validation is to review DRGs without complication or comorbidity that have a length of stay (LOS) greater than or equal to the geometric mean length of stay (GMLOS). These charts will be reviewed to identify conditions missed that would equate to the intensity of service provided. Reviewer will validate for principal diagnosis, secondary diagnosis, and procedures affecting or potentially affecting the MS-DRG were met per Medicare guidelines.

Missing Documents Received
Receipt of some supporting and missing documents like diagnosis report.

New Documents Received
Receipt of new documents for the case that requires the supporting documents in the form of reports.

On Hold—Pending Queries
When the document status is kept on hold, as the clarification is awaited for a raised query from the query authoring tool 304.

On Hold—No Queries
When the case is kept on hold and there are no further clarifications required.

Awaiting Reconciliation
The document is marked as Awaiting Reconciliation when the case is completed, and the hospital is waiting for the payment from the insurance company.

Continuing after the method 200 of FIG. 2, once the CDS reviews the cases, he/she marks the case as: on hold, schedules follow up review or complete, as per the status of the case. The initial review flag weightages are erased for case once the cases are marked and the initial review flag is retained for future reference. Besides, for the follow up review cases, these are prioritized based on the flag scores, sequenced from the highest to the lowest. After each follow-up review, flags are automatically reset to On Hold—Pending Queries or On Hold—No Queries. These markings are customizable. The follow up flags may then be re-tagged/re-marked based on case update like reconciliation and prioritized for review as per the case. Accordingly, the CDS completes case once he/she is done with the review.

The system and the method 200 explained above would be better understood with help of the following examples:

### Example 1

Assigning the initial review flag weightages to the different parameters by the hospital admin. The weightage is assigned on a scale of 0 to 10. Let us take an example to understand it further. The following are the weightages assigned to the plurality of parameters:

| Parameters | Weightage |
|---|---|
| DRG Impacting Query Opportunity | 10 |
| Mortalities with ROM <3 | 2 |
| Quality Impacting Query Opportunity | 10 |
| Target Chief Complaint/Admitting Dx | 9 |
| Clinical Validation (Missing Dx) | 9 |
| Clinical Validation (Missing evidence) | 9 |
| PSI Flag | 10 |
| All mortalities | 9 |
| No CC/MCC | 8 |
| 30-day readmission | 9 |
| Denials | 8 |
| Target DRG | 8 |
| Target PDx | 7 |
| Assigned by coding | 9 |
| Assigned by quality | 9 |
| standard review (no flags) | 0 |

If the hospital admin chooses to use the following parameters and weightages, please see the table below for further information. Weightages to the parameters assigned by Hospital 1 are mentioned below:

| Parameters | Weightage |
|---|---|
| DRG Impacting Query Opportunity | 10 |
| Flagged for Mortality with ROM <3 | 10 |
| Quality Impacting Query Opportunity | 9 |
| 30-day readmission | 9 |
| PSI Flag | 10 |
| No CC/MCC | 7 |
| Denials | 8 |
| Target DRG | 6 |

Let us understand the ranking of these cases with the following example. The weightages are assigned as per the above tabular form. The weightages are assigned as per the requirement by the hospital administration.

Case 1:

Flagged for DRG impacting query (10)

Flagged for 30-day readmission (9)

Total Score: 19

Case 2:

Flagged for Mortality with ROM<3 (10)

Flagged for PSI (10)

Total Score 20

Case 3:

Flagged for DRG impacting query (10)

Denials (8)

Total Score 18

Case sequence in the Initial Review worklist would be Case 2, Case 1, Case 3

The following are the parameters used for the follow up review and the number depicted against each parameter denotes the flag score.

Follow Up Review Case Prioritization

| | |
|---|---|
| Patient Expired | 10 |
| Discharged with pending queries | 10 |
| New DRG Impacting Query Opportunity | 9 |
| New Quality Impacting Query Opportunity | 9 |
| Scheduled for Today | 8 |
| DRG Mismatch | 7 |
| LOS > Working GMLOS | 7 |
| Missing documents received | 6 |
| New documents received | 5 |
| On Hold - Pending Queries | 0 |
| On Hold - No Queries | 0 |
| Awaiting Reconciliation | 0 |

The following are examples of cases for the follow up review:

Case 1

Missing Documents Received (6)

Total Score: 6

Case 2

Patient Expired (10)

DRG Mismatch (7)

Total Score: 17

Case 3

Query Responded (9)

LOS>Working GMLOS (7)

Total Score 16

Sequence in the Follow up Review worklist would be Case 2, Case 3, Case 1.

For the follow up review case, there are similar steps occurring. As the name suggests, follow up reviews are the cases wherein the updates are done after the initial review step. The sequencing of the cases is done as per the number of flags assigned the parameters. After every follow up review, the review flags reset to On Hold—Pending Queries or On Hold—No Queries. Later, the follow up flags are updated to reconciliation and prioritized for review as the updates happen.

Here, the present invention utilises the ‘query authoring tool 304’ connected with the computer system 102. The Query authoring tool 304 is the one where the user 306 can build query on the interface and assign weightage to each component of query. In order to reduce the gap between users 306 and the semantic web, the users 306 are provided with the ability of querying and visualizing the existing knowledge available in the ontological media repository. To this end, there is a low-abstraction-level (what you see is what you get) authoring tool supporting end user 306 and automatic customization of the retrieved information. Any user with no programming or query skills can freely manipulate high-level representations of knowledge obtained from previous queries or simply from the beginning.

Herein, the query authoring tool 304 is used to reduce the operating gap between the user 306 and the interface. This is a user-friendly tool wherein the user with no prior knowledge on the specific ontology or programming query language skills can be used to customize the query according to the information needed. This is the tool wherein the user 306 has the flexibility to modify the query to get the desired outcome.

The computer system 102 is designed to automatically generate confidence score of a particular query for the given case. The computer system 102 uses the different modules and techniques to generate confidence score based on different parameters. The machine learning pipeline is built starting from the point of collecting data to the point of generating confidence score. The module includes the following components such as, but not limited to, Collector module, Pre-processor/Cleaner, Model Builder, Score Generator etc.

The Collector module collects all required data of the case and query from the production environment. The collector module is created in such a manner that it automatically directs live stream data to the automated computer system 102. The collector module is configurable such that it depends on requirement and the data flow can be turned on or off. The data preparation includes establishing the correct data collection mechanism(s). All those case and query related data are transferred to pre-processor module.

This refers to the transformations applied to the data before feeding it to the algorithm. Some specified Machine Learning model needs information in a specified format, for example, Random Forest algorithm does not support null values, therefore, to execute random forest algorithm null values have to be managed from the original raw data set. Another aspect is that data set should be formatted in such a way that more than one Machine Learning and Deep Learning algorithms are executed in one data set, and the best out of them is chosen. So, the pre-processor module of the computer system 102 is used to convert the raw data into a clean data set and feasible to machine learning models. Some of the techniques in this module include Rescale Data, Binarize Data, Standardize Data, Decompose data, Data cleaning and Data Sampling.

Further, the word “module,” as used herein in the specification, refers to logic embodied in hardware or firmware, or to a collection of software instructions, written in a programming language, such as, for example, Java, C, or assembly. One or more software instructions in the modules may be embedded in firmware, such as an EPROM. It will be appreciated that modules may comprised connected logic units, such as gates and flip-flops, and may comprise programmable units, such as programmable gate arrays or processors. The modules described herein may be implemented as either software and/or hardware modules and may be stored in any type of computer-readable medium or other computer storage device.

Further, while one or more operations have been described as being performed by or otherwise related to certain modules, devices or entities, the operations may be performed by or otherwise related to any module, device or entity. As such, any function or operation that has been described as being performed by a module could alternatively be performed by a different server, by the cloud computing platform, or a combination thereof.

It should be noted that where the terms “server”, “secure server” or similar terms are used herein, a communication device is described that may be used in a communication system, unless the context otherwise requires, and should not be construed to limit the present disclosure to any particular communication device type. Thus, a communication device may include, without limitation, a bridge, router, bridge-router (router), switch, node, or other communication device, which may or may not be secure.

Further, the operations need not be performed in the disclosed order, although in some examples, an order may be preferred. Also, not all functions need to be performed to achieve the desired advantages of the disclosed system and method, and therefore not all functions are required.

The terms and descriptions used herein are set forth by way of illustration only and are not meant as limitations. Examples and limitations disclosed herein are intended to be not limiting in any manner, and modifications may be made without departing from the spirit of the present disclosure. Those skilled in the art will recognize that many variations are possible within the spirit and scope of the disclosure, and their equivalents, in which all terms are to be understood in their broadest possible sense unless otherwise indicated.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to be providing broadest scope of consistent with the principles and the novel and inventive features disclosed or suggested herein. Accordingly, the invention is anticipated to hold on to all other such alternatives, modifications, and variations that fall within the scope of the present invention and appended claims.

1. A computer system for worklist prioritization for Clinical Documentation Improvement (CDI) in medical coding, the computer system comprising:
a memory unit configured to store machine-readable instructions; and
a processor operably connected with the memory unit, the processor obtaining the machine-readable instructions from the memory unit, and being configured by the machine-readable instructions to:
receive one or more cases from an admin computing device associated with a hospital administration, wherein each of the one or more cases is assigned a predetermined weightage to a corresponding plurality of parameters involved in each case;
generate a confidence score of each of the one or more cases to validate the one or more cases and the predetermined weightage assigned to a corresponding plurality of parameters involved in each case;
add the predetermined weightages of each of the one or more cases based on the confidence score;
provide the one or more cases in a sequence based on a sum of predetermined weightages of each of the one or more cases from highest to lowest, the highest being indicative of a high priority case;
mark and schedule the one or more cases in the generated sequence for a CDI Specialist (CDS) for review and take up based on the priority level for query generation.

2. The computer system as claimed in claim 1, wherein for generating the confidence score, the processor is further configured to:
establish a secure interface two-way channel for data transfer between the computer system and the admin computing system;
receive data related to the one or more cases from the admin computing device using the secure interface two-way channel;
segregate the data into text data and demographic data using a HL7 parser, the text data being unstructured patient-oriented clinical data;
send the demographic data to an application database that stores all data of the one or more cases in one place from where a connected web service fetches information to send and receive client specific data;
convert the text data using Natural Language Programming (NLP) from the unstructured data into structured data;
build a query module using a query parser by receiving the text data from the NLP and a query authoring tool operated by a user, the query module being used to validate the one or more cases;
pass the data from the query parser through a scheduler which is defined by the user and/or set of algorithms whenever a predetermined set of conditions is met to prioritize the CDI worklist; and
receive the parsed query from the query parser and the data from the web service at a CDI worklist prioritization module, to generate the confidence score based on a defined algorithm.

3. The system as claimed in claim 1, wherein the review is selected from an initial review and a follow up review.

4. The system as claimed in claim 3, wherein the plurality of parameters for the initial review are selected from one or more of DRG Impacting Query Opportunity, Risk of mortality, Quality Impacting Query Opportunity, Target Chief Complaint/Admitting Diagnosis, Clinical Validation (Missing Diagnosis and missing evidence), PSI Flag, All Mortalities, No Major Comorbidity/Complication (MCC), 30-day readmission, Denials, Target Diagnosis Related Group (DRG), Target Principal/Primary Diagnosis, Assigned by Coding, Assigned by Quality and standard review.

5. The system as claimed in claim 3, wherein the plurality of parameters for the follow-up review are selected from one or more of Patient Expired, Discharged with pending queries, Query Responded, New DRG Impacting Query Opportunity, New Quality Impacting Query Opportunity, Scheduled for Today, DRG Mismatch, geometric mean length of stay (GMLOS), Missing documents received, New documents received, On Hold—Pending Queries, On Hold—No Queries and Awaiting Reconciliation.

6. The system as claimed in claim 1, wherein the predetermined weightages are provided on a scale of 1 to 10, wherein 10 is highest & indicative of higher priority.

7. A method for worklist prioritization for Clinical Documentation Improvement (CDI) in medical coding, the method comprising:
receiving one or more cases from an admin computing device associated with a hospital administration, wherein each of the one or more cases is assigned a predetermined weightage to a corresponding plurality of parameters involved in each case;
generating a confidence score of each of the one or more cases to validate the one or more cases and the predetermined weightage assigned to a corresponding plurality of parameters involved in each case;
adding the predetermined weightages of each of the one or more cases based on the confidence score;
providing the one or more cases in a sequence based on a sum of predetermined weightages of each of the one or more cases from highest to lowest, the highest being indicative of a high priority case;
marking and scheduling the one or more cases in the generated sequence for a CDI Specialist (CDS) for review and take up of the one or more case based on the priority level for query generation.

8. The method as claimed in claim 7, wherein for generating the confidence score, the method further comprises the steps of:
establishing a secure interface two-way channel for data transfer between the computer system and the admin computing system;
receiving data related to the one or more cases from the admin computing device using the secure interface two-way channel;
segregating the data into text data and demographic data using a HL7 parser, the text data being unstructured patient-oriented clinical data;
sending the demographic data to an application database that stores all data of the one or more cases in one place from where a connected web service fetches information to send and receive client specific data;
converting the text data using Natural Language Processing (NLP) from the unstructured data into structured data;
building a query module using a query parser by receiving the text data from the NLP and a query authoring tool operated by a user, the query module being used to validate the one or more cases;
passing the data from the query parser through a scheduler which is defined by the user and/or set of algorithms whenever a predetermined set of conditions is met to prioritize the CDI worklist; and
receiving the parsed query from the query parser and the data from the web service at a CDI worklist prioritization module to generate the confidence score based on a defined algorithm.

9. The method as claimed in claim 7, wherein the review is selected from an initial review and a follow up review.

10. The method as claimed in claim 9, wherein the plurality of parameters for the initial review are selected from one or more of DRG Impacting Query Opportunity, Risk of mortality, Quality Impacting Query Opportunity, Target Chief Complaint/Admitting Diagnosis, Clinical Validation (Missing Diagnosis and missing evidence), PSI Flag, All Mortalities, No Major Comorbidity/Complication (MCC), 30-day readmission, Denials, Target Diagnosis Related Group (DRG), Target Principal/Primary Diagnosis, Assigned by Coding, Assigned by Quality and standard review.

11. The method as claimed in claim 9, wherein the plurality of parameters for the follow-up review are selected from one or more of Patient Expired, Discharged with pending queries, Query Responded, New DRG Impacting Query Opportunity, New Quality Impacting Query Opportunity, Scheduled for Today, DRG Mismatch, geometric mean length of stay (GMLOS), Missing documents received, New documents received, On Hold—Pending Queries, On Hold—No Queries and Awaiting Reconciliation.

12. The method as claimed in claim 7, wherein the predetermined weightages are provided on a scale of 1 to 10, wherein 10 is highest & indicative of higher priority.
